(19) United States
(12) Patent Application Publication
(10) Pub. No.: US 2024/0264162 A1
(43) Pub. Date: Aug. 8, 2024

(54) USE OF PHOSPHO-KERATIN 8 AS A BIOMARKER FOR DETECTING DIVIDING CANCER CELL

(71) Applicant: KOC UNIVERSITESI, Istanbul (TR)

(72) Inventors: Nurhan SICAKKAN OZLU, Istanbul (TR); Busra HARMANDA, Istanbul (TR)

(73) Assignee: KOC UNIVERSITESI, Istanbul (TR)

(21) Appl. No.: 18/566,649

(22) PCT Filed: Jun. 3, 2022
(86) PCT No.: PCT/TR2022/050523

§ 371 Date: Dec. 3, 2023

(30) Foreign Application Priority Data
Jun. 3, 2021 (TR) 2021/009156

(51) Int. Cl. G01N 33/574

(52) U.S. Cl. CPC G01N 33/57496; G01N 2333/4742; G01N 2440/14

(57) ABSTRACT

A biomarker and a method of using the biomarker for the detection of dividing cancer cells specifically in the cytokinesis phase, based on the Keratin 8 S34 phosphorylation in dividing cancerous cells are provided. The method includes obtaining a biological sample and preparing a biological extract from the biological sample; determining the presence of the biomarker in the biological extract by qualitative and quantitative methods; where the biomarker is an indicator configured for indicating the dividing cancerous cell in the cytokinesis phase.

### CROSS REFERENCE TO THE RELATED APPLICATIONS

This application is the national phase entry of International Application No. PCT/TR2022/050523, filed on Jun. 3, 2022, which is based upon and claims priority to Turkish Patent Application No. 2021/009156, filed on Jun. 3, 2021, the entire contents of which are incorporated herein by reference.

### TECHNICAL FIELD

The present invention relates to a biomarker and use of the biomarker for detecting dividing cancer cells, based on the keratin 8 phosphorylation specific to cytokinesis phase of diving cells.

### BACKGROUND

Cytoskeleton consists of three fundamental structures: microtubule, actin and intermediate filament, and has many vital cellular functions. The roles and regulations of microtubules and actin filaments, two major components of the cytoskeleton, during cell division are well characterized. Particularly, due to the important role of the microtubules in the cell division, different anti-mitotic drugs targeting microtubules are developed for use in the cancer therapies. However, these drugs cause serious side effects for the patient since they affect both the cancerous cell division and normal cell division.

Besides, there are limited studies about the roles and regulations of the intermediate filaments, the other member of the cytoskeleton, during cell division. Particularly, keratins, constituting a large class of intermediate filaments, are expressed by more than 50 genes. Intracellular keratin expression varies according to the cell type, differentiation stage and functional status. Unlike microtubules and actin, a wide range of keratin proteins exhibit cell-type specific expression pattern. Due to the fact that these expressions differentiate in the cancer cells, keratins are commonly used as biomarkers in the prognosis and diagnosis of different carcinoma. Since they exhibit cell type-specific expression pattern, keratins have been commonly used for many years as immunohistochemical marker for identification of tumor tissue origin and pathological diagnosis [1,2]. Keratins undergo multiple posttranslational modifications and one of the most prominent modifications is phosphorylation. Phosphorylation of keratins regulate their solubility and function in the cells [3,4].

Cell division is one of the most fundamental and vital events that is performed by the living organisms to transfer their genetic information to the next generations. Mitotic division consists of prophase, prometaphase, metaphase, anaphase, telophase and cytokinesis stages. In prophase and prometaphase, microtubule based spindle assemble and at metaphase chromosomes are aligned at the metaphase plane where spindle microtubules are attached to the chromosomes via kinetochore structures. Chromosomes are segregated to the opposite poles in anaphase and telophase stages. Cytokinesis, including anaphase and telophase, is the cytoplasmic division to create two daughter cells. Dramatic changes are observed in the biochemistry of the cells during cell division and these changes are completed in a short time that only lasts about an hour after a cell enters mitosis. It is quite challenging to differentiate these phases without using proper biochemical probes. Due to the close relationship between the chromosome condensation and Histone 3 Serine 10 phosphorylation, Histone 3 Serine 10 phosphorylation is used as a mitotic marker. However, there is no specific biomarker for cytokinesis [5].

Mistakes during cell division can cause cell death, viability reduction and malignant diseases such as cancer [6]. Cell division has been studied extensively for more than a century [7]. From yeast cells to the mammalian cells, conserved division mechanisms are relatively well understood. The genome-wide functional analyses; genomic, transcriptomic and proteomic studies provided a list of all genes in evolutionarily conserved pathways which play role in cell division. In a review article we summarized a wide range of proteomic studies that focus on cell division [8]. Mitotic kinases are the main regulators of cell division. Aurora B kinase is one of the master mitotic kinases that specifically play roles in chromosome segregation and cytokinesis. Inhibitors developed against aurora kinase are at a pre-clinical stage in the cancer treatment [9]. There are many potential drugs targeting kinases to be used in cancer therapies. However, since they also affect the normal dividing cells, they have serious side effects. This limits the use of kinase inhibitors in cancer therapies. However, if the kinase-substrate interaction varies according to the cell type, it may have a cancer-specific treatment potential. Particularly, the antibody, which we have developed against the phosphorylation of the keratin, has the potential to be used for only in epithelial origin but not in in hematological lineages where Keratins do not express.

It is aimed to efficiently monitor the chemotherapeutic effect of anti-mitotic drugs, which are often used in the treatment of cancer, with the detection of the biomarker that detects the variant of the keratin, often used in different cancer types, during division. Many anti-mitotic drugs such as “vincristine” and “taxanes (paclitaxel-Taxol and docetaxel-Taxotere)” are widely used in the treatment of different types of cancer to stop progression of the cell in mitosis and to keep it in mitosis [10]. However, the response of patients to such drugs differs and cannot be predicted. Although the reason of this difference is not known exactly, the patient's resistance to the drug is one of the common responses [11]. For this reason, developing reagents that only recognize the cells kept in the mitosis in normal and tumor tissue cells will be highly important for both understanding the anti-mitotic cancer chemotherapy, and efficiently monitoring the response of the patient to the treatment and optimizing the treatment strategy. Thereby, it will be enabled that the response to the anti-mitotic cancer chemotherapy and the drug dosage are determined individually, and personalized medicine approach will be realized. The biomarker we detected in keratin has the potential to be a general biomarker for the cytokinesis stage since it detects the transition stage of the cells from mitosis to cytokinesis.

Keratins are important constituents of the cytoskeleton as intermediate filaments. Their most important features that distinguish them from cytoskeletal proteins such as tubulin and actin are their heterogeneous expressions in the cells. These heterogeneous expressions have enabled them to be used as biomarker in the diagnosis of a variety of different cancer types.

The International patents numbered WO2018027091A1 and WO20150221346A1, known in the state of art, disclose that Keratin 17 expression is used for the detection of bladder and pancreatic cancer and the K4 and K17 expression is used for the detection of neck cancer.

The International patent numbered WO2014138183A1, known in the state of art, discloses that vimentin, which is another intermediate filament, has a specific biomarker feature for the mesenchymal and epithelial-mesenchymal transformed circulating tumor cells.

K7/K₂O expression is a biomarker for lung and ovarian cancer. Keratins 8 and 18 are used for the classification of renal cell carcinomas. Keratin biomarkers used for the detection of cancerous cell for diagnosis purpose are determined by the combinations of different keratin expressions depending on the cancer type.

Since they exhibit cell type-specific expression pattern, keratins have been commonly used for many years as immunohistochemical marker for identification of tumor tissue origin and pathological diagnosis. Most of the adenocarcinomas express simple epithelial keratins (K8, K18, K19), on the other hand K7 and K₂O expressions vary. The combinations of keratin expression are of great importance for diagnosing different cancers. While K₂O expression is positive and K7 expression is negative in colorectal adenocarcinoma; K7 expression being positive and K₂O expression being negative in ovarian, endometrial and lung adenocarcinoma are the characteristic features of tumors. [1]. In addition to these, the characteristic keratin expressions of other cancer types are shown in Table 1. In a recent study, it is identified that K8 phospho S73 can be a possible biomarker for low Beclin 1 expression in breast cancer. Low Beclin 1 expression allows the defective autophagy in breast cancer to be understood [12].

TABLE 1
Common keratins used as diagnosis marker
in different epithelial malignancies.

| Cancer | | | |
|---|---|---|---|
| type | Keratin type | Cancer type | Keratin type |
| Skin | K1, K4-K6, K10, | Mesothelioma | K5, K7, K8, K18, K19 |
| | K13-K19 | | |
| Liver | K8, K18, K19 | Breast | K5, K5-K8, K14, |
| | | | K17-K19 |
| Biliary | K7, K8, K18-K20 | Stomach | K7, K8, K18-K20 |
| tract | | | |
| Colon | K7, K8, K18-K20 | Kidney | K7, K8, K18, K19 |
| Ovary | K7, K8, K18, K19 | Pancreas | K7, K8, K18-K20 |
| Prostate | K8, K18, K19 | Uterus | K5, K7, K8, K18, K19 |
| Lung | K7, K8, K18, K19 | Cervix | K4-K8, K10, K13-19 |

In addition to the importance of keratins and modified keratins in prognosis and diagnosis, the present invention detects dividing cancer cells unlike the other studies. Until now, there was no keratin biomarker used for the dividing cancer cells. The advantage of the biomarker of the present invention over other markers is that it detects different cell cycle phase of cancer.

### SUMMARY

The present invention relates to a biomarker that will enable the detection of the dividing cancer cells depending on the keratin morphology and a method for using the biomarker.

Main objective of the present invention relates to a biomarker that will enable the detection of the dividing cancer cells based on keratin 8 phosphorylation specific to cytokinesis phase of dividing cells and a method for using the biomarker.

The present invention relates to a novel method that can be used for enabling the detection of dividing cancer cells and it also aims to enable efficient monitoring of chemotherapeutic effect of the anti-mitotic drugs that are often used in the treatment of cancer.

The present invention relates to a cancer diagnosis method to be used for prognosis and diagnosis with the antibody which is developed for recognizing the Keratin 8 phospho S34 (serine 34 region) phosphorylation.

The invention is based on the finding that keratin 8 protein produced by mitotically dividing cancer cells can be a marker for the detection of cancer cells.

A form of the keratin 8 protein that will only detect the dividing cancerous cells has been discovered with the conducted study and a reagent has been developed against this form.

The fact that cancer cells have continuous cell division feature increases the potential of the developed reagent in cancerous cell detection. The fact that it is possible to diagnose cancer using phospho Keratin 8 in dividing cells brings novelty to this field different from the previous literature information.

The biomarker discovered with the present invention and its method of use developed together therewith have provided novelty and ease of use for the detection of cancerous cell in routine screening in clinics by detecting the cells in cytokinesis phase, which is the shortest phase during cell division.

Keratin biomarkers used for the detection of cancerous cell for diagnosis purpose are determined by the combinations of different keratin expressions depending on the cancer type. By means of the present invention, the detection of cancerous cells is performed only depending on the presence of phosphorylation in the Serine 34 region of keratin 8 protein.

The present invention has the potential to be used in various fields. The invention can be used by the scientists studying in the field of Molecular Biology and Cell Biology in cell division and particularly cytokinesis research, by the pathologists in the hospitals in tissue analyses, and the oncologists in monitoring the response of anti-mitotic chemotherapies to the treatment.

### BRIEF DESCRIPTION OF THE DRAWINGS

FIGS. 1A-1B show the study comprising specific binding of Keratin Phospho S34 antibody.

FIG. 2 shows the study comprising Keratin 8 Phospho S34 antibody staining in the Hela S3 cell where Keratin 8 gene is silenced.

FIG. 3 shows the study illustrating the localization of Phospho S34 Keratin 8 in different cell cycle stages in Hela S3 cell.

### DETAILED DESCRIPTION OF THE EMBODIMENTS

The said invention is the discovery of a biomarker for the detection of dividing cancerous cells, as well as a method comprising the detection of dividing cancerous cells by means of the said biomarker. Prognosis, diagnosis of dividing cancerous cells and monitoring of these cancer cells can be performed by means of this method.

The said invention is a method for detecting the dividing cancerous cells in a biological sample in the presence of a biomarker, comprising the following steps:
obtaining a biological sample and preparing a biological extract from the sample;
determining the presence of biomarker in the said biological extract by qualitative and quantitative methods;
wherein the biomarker is an indicator that there is a dividing cancerous cell in the cytokinesis phase.

The term “biological sample” or “sample” as used herein is the biological sample that means any one of the cancerous or healthy (non-cancerous) cells, tissues, or organs. The sample can be obtained in any manner (e.g., biopsy) known by a person skilled in the art. The sample can contain single or different type of cell, cell section, tissue or organ and the sample can be collected from any part of the subject. The sample can be fresh, frozen, or pre-processed (e.g., paraffin-embedded tissues fixed in formalin).

In a preferred embodiment of the invention, the cancer types whose detection, prognosis or monitoring are performed using biomarker of the present invention are the cancer types comprising keratin 8 protein and/or mRNA expression and these can be collected from the liver, biliary tract, colon, ovary, prostate, lung, mesothelioma, breast, stomach, kidney, pancreas, uterus, or cervix regions as sample examples. In a preferred embodiment of the invention, the cancerous cells, which are detected by the method for detecting the dividing cancerous cells using the biomarker of the present invention, are of the cancer types that are known to comprise keratin 8 protein and/or mRNA expression.

The term “subject” or “patient” as used herein refers to any mammal. The definition “a dividing cancerous cell” as used herein more particularly emphasizes the dividing cancer cell in the cytokinesis phase.

The Aurora B protein is one of the master kinases that plays role in the mitosis and cytokinesis stages of cell division. The expression of Aurora B protein shows an increase in cancerous cells. It has been found in previous studies that the cytoskeleton proteins are regulated by Aurora B kinase in mitosis and cytokinesis stages [13]. In line with this evidence, the effect of Aurora B protein on Keratin 8 protein has been investigated in the said invention. The changes of Keratin 8 during cell division were observed by using small molecule Aurora B inhibitors. In accordance with these observations, a mass spectrometry-based proteomic methods were employed in order to detect Keratin 8 phosphorylation sites regulated by Aurora B. First, Aurora B dependent phosphorylation sites of Keratin 8 were investigated in the preliminary study. For this purpose, in vitro kinase assays were performed to confirm the kinase-substrate interaction.

As a result of in vitro kinase assays, compared to the negative control, an increase in the phosphorylation of the keratin 8 S34 (serine 34) region in the presence of Aurora B was observed. By confirming the kinase-substrate interaction of Aurora B-Keratin 8, it has been discovered in the said invention that serine 34 residue of keratin 8 is the phosphorylation site of Aurora B kinase particularly depending on cytokinesis phase.

The biomarker mentioned in the said invention is the phosphorylated (Phospho S34 Keratin 8) form of the Serine 34 region of keratin 8 protein which is an indicator that it is a dividing cancerous cell in the cytokinesis phase.

In the present invention, in the step of obtaining a biological sample and preparing a biological extract from the sample, the sample is fragmented to isolate the cells or cell section, isolated cells are catabolized in a lysis solution, proteins are isolated from the lysis solution, the proteins are fragmented into peptides preferably by incubating the isolated proteins in a digestion solution comprising trypsin and the peptides are purified from the resulting mixture.

In the said invention, in the step of determining the presence of biomarker in the biological extract by qualitative and quantitative methods, these methods include any one or more of immunohistochemistry, immunofluorescence, mass spectrometry, ELISA, protein microarrays and western blotting methods. The method used in the present invention is any method known in the art for the qualitative and quantitative detection of the presence of biomarker and it shall not be limited to these listed methods.

In the present invention, keratin 8 which is phosphorylated from the S34 region is an indicator that the sample contains dividing cancerous cell in the cytokinesis phase. This indication means the presence of phospho S34 Keratin 8, which increases at the cleavage furrow of the dividing cancerous cell, and which can be determined qualitatively and quantitatively.

An increase in the amount of phospho S34 Keratin 8 in said sample is detected preferably by comparing it with that of a control sample that does not contain cancer cells.

In the said invention, in the step of determining the presence of biomarker by qualitative and quantitative methods, determining whether the extracted or isolated keratin 8 protein is phosphorylated or not is performed in the presence of a reagent. This said reagent recognizes the Serine 34 region which is phosphorylated on Keratin 8 protein.

In the present invention, in the step of determining the presence of biomarker by qualitative and quantitative methods, said reagent is an antibody, more particularly the reagent is a type of anti-keratin 8 antibody. In the present invention, this antibody is specific to Keratin 8 protein which is phosphorylated from serine 34 region, and it recognizes (binds to it) the presence of phospho S34 keratin 8 in the detection of cancerous cell. The mentioned antibody can be any one of antibody types known in the art and it is for recognizing the S34 phosphorylation in Keratin 8 protein.

Within the scope of invention, the process of detecting the presence of phospho S34 Keratin 8 by means of the antibody is performed by any one of the above mentioned methods for detecting antigen-antibody reaction.

The present invention also relates to methods and kits for identifying a subject with cancer by determining the presence of phospho S34 keratin 8 qualitatively and quantitatively in a sample and particularly for detecting the cells in cytokinesis phase.

The following examples are to better illustrate the subject of the invention and the subject of the invention is not limited to these examples.

### EXAMPLES

### Example 1—In Vitro Phosphorylation of Keratin 8

First, Aurora B kinase dependent phosphorylation sites of Keratin 8 were investigated in our preliminary study. For this purpose, in vitro kinase assays were performed to confirm the kinase-substrate interaction.

For this assay, unmodified Keratin 8 protein fused with GST that is expressed in bacteria was purified using GST beads and used as substrate in the assay. Briefly, Keratin 8-GST protein was cloned into pGEX-6P-1 vector and grown in E. coli bacteria cells. The precipitated bacteria were dissolved with lysis solution (100 mM TrisCl pH 8, 5 mM EDTA, 5 mM DTT and 1× Protease inhibitor) and homogenized. According to our experiences, Keratin forms insoluble aggregates (Inclusion Body) in bacteria. For this reason, after centrifugation and washing, the pellet was dissolved using extraction solution (8M Urea, 5 mM DTT, 2 mM EDTA, 10 mM TrisCl pH 8) and homogenized. After the solution was centrifuged at 100.000 rpm at 4° C. for 1 hour, the supernatant was collected as recombinant protein (Keratin 8-GST) isolated from inactive insoluble aggregates. To enable correct folding of the isolated Keratin 8 recombinant protein, it was mixed and incubated in dialysis solution (25 mM Hepes pH 7.4, 100 mM KCl, 5 mM MgCl2, 0.5 mM EGTA) in a dialysis bag at 4° C. Isolation of Keratin 8-GST recombinant protein from bacteria was confirmed by Coomassie Brilliant Blue staining method. Aurora B kinase was purified together with INCENP. Active Aurora B interacts with INCENP protein. The purified Keratin 8-GST is incubated with Aurora B in the presence of ATP and a sample without Aurora B was prepared as a negative control. At the end of the kinase reaction, the obtained Keratin 8-GST was subjected to the phospho analysis using mass spectrometry and the phosphorylation points were determined. Mass spectrometry-based proteomics studies is a method often used for the detection of biomarkers. To prevent the proteins from denaturing and accumulating, DTT (C4H10O2S2) was added and then the proteins were alkylated with IAA (C2H4INO). It was enabled that the proteins were fragmented into peptides by incubating with trypsin enzyme. The obtained peptides were cleaned with Sep-Pak column. After the obtained peptides were analyzed in mass spectrometry, they were analyzed with Skyline program.

With the in vitro kinase assay, compared to the negative control, an increase in the phosphorylation of the Keratin 8 S34 region in the presence of Aurora B was observed. By confirming the kinase-substrate interaction of Aurora B-Keratin 8, Serine 34 region of Keratin 8 was discovered as the phosphorylation of Aurora B kinase particularly depending on cytokinesis phase.

### Example 2—Developing Antibody Against Phospho S34 Keratin 8 and Determining the Study Conditions

To investigate the regulation of Keratin 8 S34 phosphorylation in cell division in biological samples, the companies producing customer-specific antibody were contracted for producing Keratin 8 Phospho S34 antibody. Davids Biotech company in Germany has made it possible to produce an antibody that recognizes the phosphorylation of Keratin 8 from rabbits. By using this antibody in immunofluorescence experiments, its change in the cell division was investigated. It was observed that phospho S34 Keratin 8 placed at the cleavage furrow especially from the anaphase stage of cancer cell division until the end of cytokinesis.

There are several important technical details of the antibody production process. First, antigen must be obtained to be able to produce phospho-specific antibodies. To obtain the antigen, as a first step, the peptide is produced in a way that the region to be recognized by antibody is in the middle. A phosphorylated version of the phospho-specific region is synthesized together with 10 mg Normal peptide of >95% purity. Phospho added peptide is conjugated with KLH. In case of failure of phospho antibody production, two rabbits were immunized with antigen. At the end of 63 days, serum is collected and antibody synthesizing clones are determined by ELISA method. In the final stage, the desired phospho antibody is purified by affinity chromatography. At this stage, phospho peptide is used as positive affinity, while negative affinity purification is ensured by using normal peptide.

The concentration of the produced antibody is 160 μg/ml. The produced antibody can be used in different techniques. It is suitable for use in western blot, immunofluorescence, immunohistochemistry, or protein precipitation experiments.

We determined in our studies that two different antibodies produced specifically detect phospho S34 Keratin 8 in western blot and immunofluorescence experiments. 0.32 μg/ml (1:500 dilution) was determined as the study concentration in Western blot experiments and 1.6 μg/ml (1:100 dilution) in immunofluorescence experiments.

### Example 3—Detection of the Positioning of Phospho S34 Keratin 8 within the Cell in the Cell Cycle

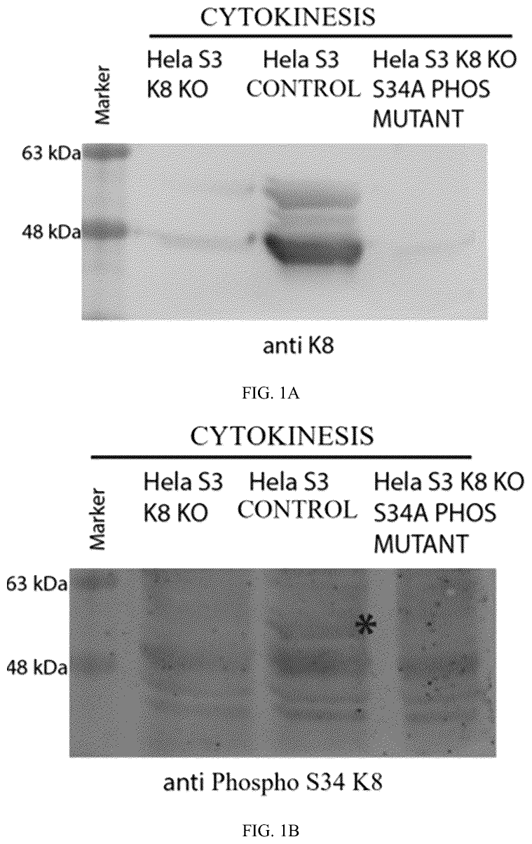

Western blot method was applied to show that the produced antibody could specifically detect the expressed phospho S34 Keratin 8 within the cell. For this, a cancer cell line whose Keratin 8 gene was blocked was used as a negative control. In addition, plasmid having Keratin 8 S34A mutation was transfected in the cancer cell line whose Keratin 8 gene was blocked. Cancer cell samples synchronized to cytokinesis were collected as positive control. (FIGS. 1A-1B)

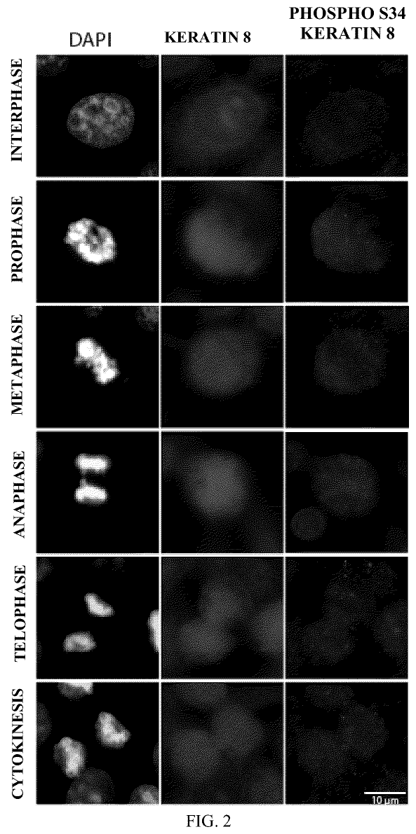

Immunofluorescence experiments were performed to detect the localization of the produced antibody within the cell during cancerous cell division. The cancerous Hela S3 (Hela S3 K8 KO) cell line whose Keratin 8 gene was silenced, was used as negative control. (FIG. 2)

When the western blot results were evaluated, it was determined that the produced antibody specifically recognized the Keratin 8 phospho S34 modification (FIGS. 1A-1B). The Keratin 8 phospho S34 band shown in the positive control matched with the Keratin 8 antibody, while the Keratin 8 phospho S34 band was observed in the negative controls.

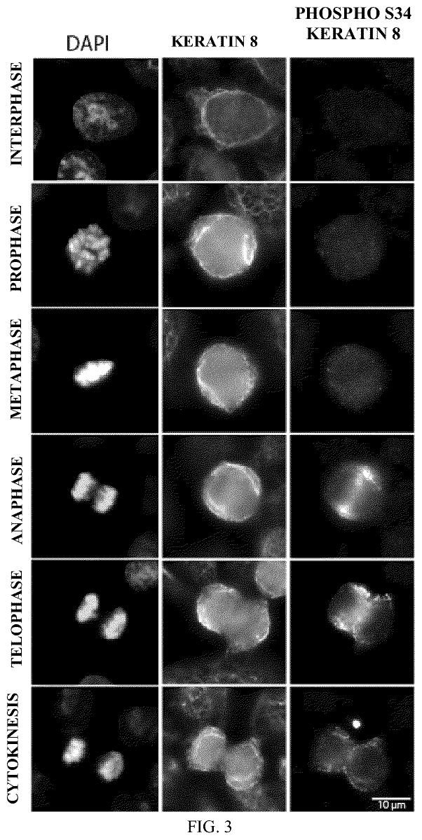

Keratin 8 and Keratin 8 Phospho S34 antibody stainings were used in the Hela cell line whose Keratin 8 gene was silenced, which was used as a negative control (FIG. 2). Immunofluorescence experiments show that the Keratin 8 S34 phosphorylation we detected was specifically localized to the cleavage furrow from the anaphase phase to the end of cytokinesis. Keratin 8 S34 phosphorylation was not observed in interphase, prophase, prometaphase, and metaphase stages (FIG. 3). These results show that Keratin 8 S34 phosphorylation particularly detects dividing cells.

Regarding these basic concepts, it is possible to develop a wide variety of applications by means of “Use of Phospho-Keratin 8 As Biomarker in the Detection of Dividing Cancer Cell” of the present invention and the invention, which is essentially as defined in the claims, is not limited to examples disclosed herein.

### REFERENCES

[1]. Moll, R., Divo, M., & Langbein, L. (2008). The human keratins: biology and pathology. Histochemistry and cell biology, 129(6), 705-733.
[2]. Karantza, V. (2011). Keratins in health and cancer: more than mere epithelial cell markers. Oncogene, 30(2), 127-138.
[3]. Chou, C. F., Riopel, C. L., Rott, L. S., & Omary, M. B. (1993). A significant soluble keratin fraction in ‘simple’ epithelial cells. Lack of an apparent phosphorylation and glycosylation role in keratin solubility. Journal of Cell Science, 105(2), 433-444.
[4]. Snider, N. T., & Omary, M. B. (2014). Post-translational modifications of intermediate filament proteins: mechanisms and functions. Nature reviews Molecular cell biology, 15(3), 163-177.
[5]. Hendzel, M. J., Wei, Y., Mancini, M. A., Van Hooser, A., Ranalli, T., Brinkley, B. R., . . . & Allis, C. D. (1997). Mitosis-specific phosphorylation of histone H3 initiates primarily within pericentromeric heterochromatin during G2 and spreads in an ordered fashion coincident with mitotic chromosome condensation. Chromosoma, 106(6), 348-360.
[6]. Mitchison, T. J., & Salmon, E. D. (2001). Mitosis: a history of division. Nature cell biology, 3(1), E17-E21.
[7]. Rieder, C. L., & Khodjakov, A. (2003). Mitosis through the microscope: advances in seeing inside live dividing cells. Science, 300(5616), 91-96.
[8]. Üretmen Kagιalι, Z. C., Şentürk, A., Özkan Küçük, N. E., Qureshi, M. H., & Özlü, N. (2017). Proteomics in cell division. Proteomics, 17(20), 1600100.
[9]. Oke, A., Pearce, D., Wilkinson, R. W., Crafter, C., Odedra, R., Cavenagh, J., . . . & Bonnet, D. (2009). AZD1152 rapidly and negatively affects the growth and survival of human acute myeloid leukemia cells in vitro and in vivo. Cancer research, 69(10), 4150-4158.
[10]. Kaestner, P., & Bastians, H. (2010). Mitotic drug targets. Journal of cellular biochemistry, 111(2), 258-265.
[11]. Kavallaris, M. (2010). Microtubules and resistance to tubulin-binding agents. Nature Reviews Cancer, 10(3), 194-204.
[12]. Kongara, S., Kravchuk, O., Teplova, I., Lozy, F., Schulte, J., Moore, D., . . . & Karantza, V. (2010). Autophagy Regulates Keratin 8 Homeostasis in Mammary Epithelial Cells and in Breast TumorsPhospho-K8 in Mammary Cells and Breast Tumors. Molecular Cancer Research, 8(6), 873-884.
[13]. Polat, A. N., Karayel, O., Giese, S. H., Harmanda, B., Sanal, E., Hu, C. K., . . . & Özlü, N. (2015). Phosphoproteomic analysis of aurora kinase inhibition in monopolar cytokinesis. Journal of proteome research, 14(9), 4087-4098.

What is claimed is:

1. A method for detecting a dividing cancerous cell in a biological sample in a presence of a biomarker, comprising following steps:
obtaining the biological sample and preparing a biological extract from the biological sample;
determining the presence of the biomarker in the biological extract by qualitative and quantitative methods;
wherein the biomarker is an indicator configured for indicating the dividing cancerous cell in a cytokinesis phase.

2. The method according to claim 1, wherein the biomarker is phospho S34 Keratin 8.

3. The method according to claim 1, wherein the step of determining the presence of the biomarker is performed by at least one method selected from the group of immunohistochemistry, immunofluorescence, mass spectrometry, ELISA, protein microarrays, and western blotting.

4. The method according to claim 1, wherein the step of determining the presence of the biomarker is performed by a reagent.

5. The method according to claim 4, wherein the reagent is an antibody.

6. The method according to claim 5, wherein the antibody recognizes a phosphorylated S34 region in a Keratin 8 protein.

7. The method according to claim 6, wherein a presence of the phosphorylated S34 region in the Keratin 8 protein is an indicator configured for indicating that biological sample comprises the dividing cancerous cell.

8. The method according to claim 7, wherein the dividing cancerous cell is one of liver, biliary tract, colon, ovary, prostate, lung, mesothelioma, breast, stomach, kidney, pancreas, uterus, or cervix regions.

9. A kit for identifying a subject with a cancer by determining presence of phospho S34 Keratin 8 in a sample, wherein the kit is performed by the method according to claim 1.

10. The method according to claim 2, wherein the step of determining the presence of the biomarker is performed by at least one method selected from the group of immunohistochemistry, immunofluorescence, mass spectrometry, ELISA, protein microarrays, and western blotting.

11. The method according to claim 2, wherein the step of determining the presence of the biomarker is performed by a reagent.

12. The method according to claim 3, wherein the step of determining the presence of the biomarker is performed by a reagent.

13. The kit according to claim 9, wherein in the method, the biomarker is phospho S34 Keratin 8.

14. The kit according to claim 9, wherein in the method, the step of determining the presence of the biomarker is performed by at least one method selected from the group of immunohistochemistry, immunofluorescence, mass spectrometry, ELISA, protein microarrays, and western blotting.

15. The kit according to claim 9, wherein in the method, the step of determining the presence of the biomarker is performed by a reagent.

16. The kit according to claim 15, wherein the reagent is an antibody.

17. The kit according to claim 16, wherein the antibody recognizes a phosphorylated S34 region in a Keratin 8 protein.

18. The kit according to claim 17, wherein a presence of the phosphorylated S34 region in the Keratin 8 protein is an indicator configured for indicating the biological sample comprises the dividing cancerous cell.

19. The kit according to claim 18, wherein the dividing cancerous cell is one of liver, biliary tract, colon, ovary, prostate, lung, mesothelioma, breast, stomach, kidney, pancreas, uterus, or cervix regions.
